(19) United States
(12) Patent Application Publication
(10) Pub. No.: US 2005/0080027 A1
(43) Pub. Date: Apr. 14, 2005

(54) Optimized production of viral vectors derived from paroviruses in packaging and production cells by hsv infection or treatment with dna methylation inhibitors

(76) Inventors: Markus Horer, Martinsried (DE); Ralf Dubielzig, Munchen (DE)

Correspondence Address: CLARK & ELBING LLP, 101 FEDERAL STREET, BOSTON, MA, 02110, US

(21) Appl. No.: 10/497,227

(22) PCT Filed: Nov. 29, 2002
(86) PCT No.: PCT/EP02/13532

Related U.S. Application Data
(60) Provisional application No. 60/334,571, filed on Nov. 30, 2001.

(51) Int. Cl. A61K 48/00; C12N 15/86

(52) U.S. Cl. 514/44; 424/93.2; 514/46; 514/49; 435/456

(57) ABSTRACT

The invention relates to uses and methods enabling the production yield of viral vectors derived from piroviruses to be improved by using DNA methylation inhibitors and/or Herpes viruses in the production of viral vectors derived from parvoviruses.

The present invention relates to compositions for increasing the yield when producing viral vectors which are derived from parvoviruses.

Genetically modified viruses have turned out to be suitable for transferring genes into mammalian cells, in particular into human cells. For this, the viruses are as a rule modified genetically in order to be able to use them as carriers (viral vectors) for transferring one or more transgenes.

Examples of currently employed viral vectors are vectors which are derived from adenoviruses, herpesviruses, retroviruses or parvoviruses, such as the adenoassociated viruses (AAVs) (Pfeifer and Verma (2001) Annu. Rev. Genomics Hum. Genet. 2:177-211).

The parvovirus family (Parvoviridae) comprises the smallest (18-26 nm) viruses, which are not enveloped by a membrane. The parvovirus genome contains a linear single-stranded DNA, with + and − strands being packaged in the same proportions. The parvovirus family is divided into two subfamilies, i.e. the Parvovirinae and the Densovirinae. The Parvovirinae in turn comprised three genera, i.e. the parvoviruses, the erythroviruses and the dependoviruses. AAV belongs to the dependoviruses and is a human virus which is either integrated into the genome in the form of a provirus or gives rise to a lytic infection. For this reason, AAV is of interest as a general vector for transducing mammalian cells. A large number of AAV serotypes are currently known, e.g. AAV-1, AAV-2, AAV-3, AAV-4, AAV-5, AAV-6, AAV-7 and AAV-8 (Gao G-P et al. (2002) PNAS 99:11854-9). It is to be expected that additional AAV serotypes will be isolated in the future. AAV-2, for example, contains a linear single-stranded DNA which is approx. 4.7 kilobases (kb) in length. The viral particles, which are composed of three viral proteins, i.e. VP1, VP2 and VP3, contain a strand of viral DNA which possesses either the one polarity (+) or the other (−). Examples of AAV-derived viral vectors are well known. Possibilities for preparing them are described subsequently. The invention also includes capsid mutants of these serotypes. Within the context of this invention, “capsid mutants” are understood as meaning that the AAV particles can contain a mutated capsid. This capsid can contain a mutation or one or more amino acids, or contain one or more deletions and/or insertions. Appropriate examples are known to the skilled person from the following literature references: WO 99/67393, Grifman M. et al. (2001) Mol. Ther. 3(6):964-75, Wu P. et al. (2000) J. Virol 74(18):8635-47, Chandler L A et al. (2000) Mol. Ther. 2(2):153-60, Hirate R K and Russell D W (2000) J. Virol. 74(10):4612-20, Girod A et al. (1999) Nat. Med. 5(12):1438, Girod A et al. (1999) Nat. Med. 5(9):1052-6 or in Bartlett J S et al. (1999) Nat. Biotechnol. 17(2):181-6.

An important point for consideration when developing suitable live vectors is that of the safety aspects connected with using said vectors in human gene therapy. For this reason, “replication-deficient” viruses are generally developed, i.e. viruses which, while being able to infect a cell and transfer the transgenes into this cell, are not able to replicate in these cells. This is accomplished, for example, by deleting genes which are important for replication of the virus, e.g. genes which encode structural proteins, and, where this is appropriate, incorporating the transgene or the transgenes in their stead. Additional genes, which compensate for the lack of the structural protein genes in the cell, are then necessary for producing the nonreplicateable viruses which are required for the gene therapy.

In general, the following genes are required for forming virus particles:
a) genes which occur naturally on the virus genome but (some of) which can no longer be present in the recombinant virus. In the case of AAV, these genes are the rep and cap genes. Within the context of this invention, nucleic acid sequences which carry these genes are designated “helper constructs”.
b) genes which occur on the genome of other viruses (what are termed “helper viruses”) or genes which occur on the genome of the cell in which the virus particles are produced. Within the context of this invention, nucleic acid sequences containing these genes are designated “helper genes”. In the case of viral vectors, these helper genes are not required for particle formation.

Within the meaning of the present invention, “helper genes” are, in particular, understood, in connection with preparing AAV-vectors, as being the genes of the AAV helper viruses and/or cellular genes whose gene products are necessary for, or promote, replication of the AAV. Examples of adenoviral helper genes are the genes E1A, E1B, E4, E2A and VA. In this connection, E1A is required for transactivating the AAV p5 promoter. The gene products E1B and E4 are used for augmenting the accumulation of AAV mRNA. The gene products E2A and VA are used for augmenting the splicing and translation of AAV mRNA. The helper genes also include herpes simplex virus (HSV) helper genes. These can, for example, be the seven replication genes UL5, UL8, UL9, UL29, UL30, UL42 and UL52. UL5, 8 and 52 form the HSV helicase primase complex, while UL29 codes the single-stranded DNA-binding protein, UL42 encodes a double-stranded DNA-binding protein, UL30 encodes the HSV DNA polymerase and, finally, UL9 encodes a protein which binds to the HSV origin of replication (see Weindler F W and Heilbronn R (1991) J. Virol. 65(5):2476-83). Using the helper virus, for example adenovirus type 5 (Ad5), instead of the individual helper genes is particularly advantageous since this comes closest to the natural situation of AAV replication in the presence of helper viruses and, as a consequence, the packaging of rAAV particles is very efficient. Examples of other helper viruses are herpesviruses or vaccinia viruses.
c) nucleic acids which contain the heterologous DNA, i.e. the DNA which is foreign to the virus and which is to be introduced into other cells using the viral vector. This DNA is as a rule flanked by endogenous viral sequences, such as ITRs, which are important for replication of DNA and for particle formation. Within the context of this invention, nucleic acid sequences which encompass the heterologous DNA together with said sequences, such as ITRs, which are endogenous to the virus, are designated “vector constructs”.

In that which follows, the preparation of viral vectors is explained in more detail taking AAV as an example:

A method for preparing relatively large quantities of rAAV particles is that of cotransfecting a eukaryotic cell with two recombinant AAV plasmids in the form of a mixture and infecting with a helper virus (Chiorini, J. A. et al. (1995) Human Gene Therapy 6:1531). The first recombinant AAV construct contains one or more transgene(s) which is/are bordered, i.e. flanked, by two ITR regions (vector constructs). The second recombinant AAV construct, i.e. the helper construct, contains the AAV genes which are required for producing the virus particles (rep and cap genes). The absence of the ITR regions in the helper construct is intended to prevent the rep and cap genes from being packaged in AAV particles and thus prevent the formation of unwanted wild-type AAV. Suitable cells which are permissive for, i.e. accessible to, both the recombinant AAV construct and the helper virus are then transfected with both AAV constructs. Examples of such permissive cells are HeLa cells. After the transfected cells have been infected with helper viruses, for example adenovirus, the AAV genes are expressed, the transgenic DNA is replicated and the recombinant AAV particles (rAAV particles) are packaged and assembled. The rAAV particles contain the transgene(s), flanked on both sides by the ITR regions, in the form of a single-stranded DNA. At the same time, the helper virus replicates in these cells, an event which, when adenoviruses are used as helper viruses, generally leads to the lysis and death of the infected cells after a few days. Some of the rAAV particles, and the helper viruses which have been formed, are released into the cell culture medium in this connection while the rest remain in the lyzed cells. An example of a review of the use of AAV as a general vector for transducing mammalian cells is that provided by Muzyczka, N. (1992) in Current Topics in Microbiology and Immunology 158:97.

A substantial disadvantage of this way of preparing rAAV particles is that helper constructs and vector constructs have to be produced for each round of preparation, something which is an expensive process under GMP conditions. In addition, plasmid transfections are steps which should be avoided in a commercial production process.

In order to avoid transfecting plasmids, packaging cell lines were developed, these cell lines containing copies of the entire AAV genome without the flanking ITRs and with rep and cap genes under the control of their natural viral promoters. In the absence of an infection with a helper virus, these promoters, i.e. P5, P19 and P40, are inactive (Inoue and Russels (1998) J. Virol. 72:7024-7031; Gao et al. (1998) Human Gene Therapy 9:2353-2362). Following infection with a helper virus, the stably transfected packaging cell line (e.g. HeLa cell line) is induced to express the AAV genes. These cell lines can be used for preparing large quantities of AAV, in particular for commercial applications (Allen et al. (1997) J. Virol. 71:6816-6822; Wang et al. (1998) J. Virol. 72:5472-5480).

The disadvantage of using the published packaging cell lines for preparing viral vectors is the fact that only one recombination event with the vector genome is required for forming wild-type AAV (rcAAV), for which reason these cell lines are not suitable for being used in gene therapy because of the immense risk involved.

New helper constructs and vector constructs, which are suitable for preparing rAAV and which are used for producing host cell lines in the form of packaging and producer cell lines, were developed in order to make it possible to produce rAAV on a large scale while at the same time essentially preventing the formation of wild-type AAV. The strategy of functionally separating the rep and cap genes was pursued for this purpose (see DE 10044348).

Viral vectors, in particular AAV viral vectors, are preferably prepared using a packaging cell, a vector cell or a producer cell (definition, see below). These cells can be dependent on, or independent of, a helper virus.

Such a packaging cell, vector cell or producer cell can be dependent on a helper virus if the AAV production requires an infection with a helper virus. However, such a packaging cell, vector cell or producer cell can also be independent of a helper virus if the AAV production does not require any infection with a helper virus. Such a packaging cell, vector cell or producer cell which is independent of a helper virus normally contains genes, which are required for inducing AAV production, under the control of an inducible promoter. These genes can be of viral or cellular origin.

Within the context of the experiments which were carried out for the purpose of developing novel helper constructs and vector constructs which were suitable for preparing rAAV, an attempt was initially made to use a cap gene expression construct in which the cap gene was under the control of the homologous P40 promoter.

The terms “natural promoter” and/or “homologous promoter” mean that the genetic unit of the promoter or of the regulatory sequence is derived from the same organism as the remainder of the unit with which it is prepared. Conversely, a “heterologous promoter” or “unnatural promoter” means that the promoter has been separated from its natural coding sequence and has been operatively linked to another coding sequence.

The use of a cap gene expression construct under the control of the P40 promoter was based on the general opinion that, in the case of AAV-2, the P40 promoter (or the corresponding promoters in the case of the other AAV serotypes) more or less on its own regulated the expression of the cap gene (Snyder R O (1999) J. Gene Med. 1:166-75). However, it was found that while these constructs brought about strong expression of the Cap protein, they were no longer regulable (i.e. constitutive) and consequently also no longer inducible by helper viruses (comparable with a heterologous promoter). As a consequence, it was not possible to stably integrate such a cap expression construct in the host cells since the constitutively expressed Cap proteins presumably had a toxic effect on the cells. Furthermore, it was evidently in the main empty capsids which were formed when this cap expression construct was used since the titers of transducing rAAVs which were achieved were comparatively low.

As described in DE 10044348, it is possible to use a HeLa cell-based packaging cell line, in which the AAV rep and cap genes are functionally separated and the cap gene is also under the control of the other homologous promoters, which are actually assigned to the rep gene, i.e. P5, P19 and P40 in the case of AAV-2, to achieve high yields of rAAV particles without replication-competent wild-type AAV (rcAAV) particles being formed at the same time. The designations P5, P19 and P40 are used for all serotypes (Xiao et al. (1999) J. Virol. 73:3994-4003; Bantel-Schaal et al. (1999) J. Virol. 73:939-47; Chiorini et al. (1999) J. Virol. 73:1309-19; Chiorini et al. (1997) J. Virol. 71:6823-33); however, in the case of AAV-4, the P5 promoter has also been termed the P7 promoter (Chiorini et al. (1997) J. Virol. 71:6823-33).

The expressions “functionally independent units” or “functionally separate” are understood as meaning that two or more genes do not overlap, with the term “gene” encompassing the corresponding promoter as well as the coding sequence. Specifically, this means, in the case of the rep and cap genes, where, in the wild-type AAV genome, the coding sequence of the rep gene overlaps with the coding sequence of the cap gene and the cap promoter (P40), that the two genes no longer overlap. This is achieved, for example, by both parts of the jointly employed coding sequence, and the P40 promoter, being duplicated (see, for example, FIG. 8). This means that the genes can be arranged in various ways in a genome. In the first place, the genes can be located at different sites in the genome, whether this means being integrated at different sites in the genome or being located on different plasmids, or a mixture of these two possibilities. In the second place, the genes can also be located alongside each other on the same DNA molecule, for example a chromosome or a plasmid, with, however, each gene being controlled by its own promoter. Such an arrangement is probable, for example, if two genes on different DNA molecules are transfected jointly. These molecules can form concatamers during the transfection, with these concatamers then integrating at one site in the genome but still forming functionally independent units.

In the described cell lines, the functionally separate rep and cap genes can be present in transiently, that is episomally, transfected form or can integrate at the same site, for example as concatamers, or at different sites in the cellular genome. The advantage of such an arrangement is that in each case at least two independent recombination events, which, at a frequency of in each case 10⁻⁷ per cell division, that is a total of 10⁻⁴, are extremely rare, would be required for reconstituting rcAAV particles. In fact, it was not possible to detect any rcAAV in a recombinant virus preparation which contains 2×10¹⁰ genomic particles.

“The stable expression” of a protein in a cell means that the DNA encoding the protein is integrated into the genome of the host cell and is therefore stably transmitted to the daughter cells during cell division. In addition, “stable expression” can mean that the DNA is present episomally and is kept stable by means of replicating independently. This is achieved, for example, by means of known, in particular viral replication systems which consist of an initiator protein (e.g. SV40 large T antigen, EBNA 1) and an origin of replication (e.g. SV40 ori, EBV oriP). Although episomes, such as plasmids, can, under particular conditions, also be transmitted to the next generation, genetic material which is present episomally in the host cell is lost more rapidly than is chromosomally integrated material. For example, it is possible to achieve the maintenance and transmission of the genetic material of interest by incorporating a selectable marker in the immediate vicinity of the polynucleotide of interest, thereby making it possible to keep the host cells, which are carrying the polynucleotide, under selection pressure.

In summary, DE 10044348 consequently described the preparation of HeLa cell-based packaging cell lines in which the AAV rep and cap genes are functionally separate and the cap gene is under the control of the homologous promoters, e.g. P5, P19 and P40. At the same time, the promoter regions of the P5, P19 and P40 promoters were, in the case of the AAV-2 helper constructs, altered by mutagenesis such that while the promoter function in regard to starting the transcription remained intact, these constructs were unable to express any functional Rep protein. In this connection, a functional Rep protein is understood as meaning that the Rep protein is able to exert the functions which are ascribed to it. While, in the present case, short Rep fragments are synthesized, these fragments are unable to assume any important functions of the Rep proteins. The skilled person is familiar with other possibilities for using mutagenesis to inactivate expression of the Rep protein, e.g. by means of inactivating the transcription start.

Thus, it was necessary to supply the two large Rep proteins Rep 68 and Rep 78, for the adenovirus-inducible transactivation of the P40 promoter, and the small Rep proteins, which are essential for the AAV packaging, from a second source (in trans or in cis). It was now possible to stably integrate these Rep expression constructs into the genome of the host cells. These constructs have the advantage that, while no toxic quantities of Rep proteins are expressed in the absence of a helper virus, the constructs ensure very strong helper virus-inducible Rep protein expression.

In general, a promoter which is operatively linked to a gene to be transcribed, but which is not necessarily located in direct spatial proximity to the gene to be transcribed, is described as being an element which is “cis” to a coding sequence. The term “operatively linked” refers to the arrangement of two or more components. Since a relationship exists between the components, they are able to exert their function in a coordinated manner. For example, a sequence which regulates transcription, or a promoter, is operatively linked to the coding sequence when the sequence which regulates transcription, or the promoter, regulates or, respectively, starts the transcription of the coding sequence. On the other hand, an element which is located on a different DNA molecule is described as being an element which is “trans” to a coding sequence.

The term “regulatory sequence” (“sequence which regulates”) is understood as meaning a genomic region which regulates the transcription of a gene to which it is linked. While, as they are described in the present invention, sequences which regulate transcription include at least one transcriptionally active promoter, they can also include one or more transcription enhancer(s) and/or transcription terminator(s).

One type of preferred helper construct for producing the preferred host cell for packaging rAAV contains nucleic acid sequences which encode at least one Rep protein, with Rep proteins being understood, for example in the case of AAV-2, as being the proteins Rep 78, Rep 68, Rep 52 and Rep 40,in particular Rep 68, Rep 52 and Rep 40, especially Rep 68 and Rep 52. The other type of preferred helper construct contains nucleic acid sequences which encode at least one of the known Cap proteins, with the Cap proteins being the proteins VP1, VP2 and VP3. The genes for these proteins, and the ITR sequences, can be isolated from wild-type AAVs, which are available generally in the form of clones. Thus, the clone pSM620 is, for example, described in Samulski et al. (1982) Proc. Natl. Acad. Sci. USA 79:2077; while the clone pAV1 is described in Laughlen et al. (1983) Gene 23:65 and the clone sub201 is described in Samulski (1987) J. Virol. 61:3096.

In a particularly suitable embodiment described in DE 10044348, expression of the Rep protein in the case of AAV-2 is controlled by the natural AAV promoter P5 while expression of the Cap protein in the case of AAV-2 is controlled by the natural AAV promoter P40, in particular by the natural AAV promoters P19 and P40, especially by the natural AAV promoters P5, P19 and P40. The same applies, in a corresponding manner, to the complementary promoters of the other AAV serotypes.

Previous experiments had shown that using heterologous promoters for expressing Rep did not provide regulation which was adequate for achieving a high yield of rAAV (Hölscher C. et al. (1994) J. Virol. 68, 7169-7177); Yang Q. et al. (1994) J. Virol. 68, 4847-4856). In a particularly preferred embodiment of DE 10044348, the Cap expression plasmid in the case of AAV-2 contains the AAV promoters P5, P19 and P40 in order to make possible an expression which is regulated in dependence both on helper virus infection or helper virus gene products and on Rep protein expression, since this arrangement best reflects the natural lytic AAV life cycle. This arrangement proved to be very suitable for achieving strictly regulated expression of Cap protein. Although numerous studies which made use of heterologous promoters for expressing large quantities of Cap protein have hitherto been published in the literature (Gao et al. (1998), see above, Grimm D. (1998) Human Gene Therapy 9:2745-2760), the natural promoters P5, P19 and P40 were used for expressing Cap within the context of this invention since it was found that the P40 promoter acts as a constitutive promoter when it is no longer in the natural environment of the other promoters P5 and P19. By using the natural AAV regulatory sequence, it was possible ensure that transcription factors which are required for expressing the cap genes in a regulated manner all find binding sites, for exerting their regulatory functions, in the natural promoter region.

In another preferred embodiment of DE 10044348, expression of the Rep protein and expression of the Cap protein in the host cell are regulated independently of each other. This approach was chosen because it was found that weak Cap expression is initially required for efficient packaging of rAAV in stable cell lines since, otherwise, high quantities of Cap have a toxic effect on the cells or else large quantities of empty capsids are formed. On the other hand, Cap must be expressed strongly at the time of packaging. A constitutive, heterologous promoter cannot fulfill these two criteria simultaneously. While this can be improved by using inducible heterologous promoters, it is in practice extremely difficult to implement the precise chronological regulation, and the strength of the Cap expression, when employing such promoters. Using the natural homologous promoters couples the expression of Cap to the activation by helper virus gene products and/or cellular helper genes, as well as Rep, and consequently regulates it chronologically exactly as in the wild-type situation.

The transcription of the nucleic acids which encode the Rep proteins and the Cap proteins is particularly advantageously terminated by the natural regulatory sequences, in particular by the natural AAV polyA signal. Just as when initiating transcription, using homologous sequences for terminating the transcription of the AAV cap and rep genes also increases the quantity of the rAAV particles which are produced by the AAV vector system.

A eukaryotic cell, preferably a mammalian cell, particularly preferably an insect cell or human cell, or a cell line, in particular HeLa cells, A549 cells, K209 cells, B50 cells or Z211 cells (the latter, see Gao G. et al. (2002) Mol. Ther. 5:644-649) is expediently used as the host cell. In principle, it is possible to use any cell or cell line which is permissive for, i.e. accessible to, the vector construct, the helper construct and, where appropriate, the helper virus. HeLa cells have proved to be particularly advantageous because the AAV P5 promoter is virtually inactive in HeLa cells and it is therefore possible to stably integrate, into their genome, a cassette for expressing the AAV Rep protein under the control of the natural regulatory elements such that the Rep protein does not have a toxic effect in these cells (Clarke et al. (1995) Human Gene Therapy 6, 1229-1341; Tamayose et al. (1996) Human Gene Therapy 7, 507-513; Inoue & Russell (1998) see above; Gao et al. (1998) see above).

Another preferred embodiment of DE 10044348 comprises a helper construct which contains nucleic acid sequences which encode at least one Rep protein, with the Rep proteins being Rep 68, Rep 52 and/or Rep 40 but not Rep 78 since it was found, surprisingly, that, together with Rep 52, Rep 40 and the three Cap proteins VP1, VP2 and VP3, the additional expression of only Rep 68 was sufficient for packaging AAV vectors. The advantage of these Rep 78-deficient helper constructs is that the largest Rep protein, which is most toxic for the packaging cells, is not expressed at all. It was furthermore found that, of the Rep proteins Rep 78 has the greatest inhibitory activity on cellular processes such as transcription. Using this helper construct can therefore increase the packaging efficiency due to the absence of Rep 78. In the natural system, both Rep 68 and Rep 78 are expressed by the P5 promoter. Using the Rep 78-deficient helper construct is also advantageous because, in adenovirus-infected cells, Rep 68 is a more powerful transactivator of the AAV promoters P19 and P40 than is Rep 78 (Hörer et al. (1995) J. Virol. 69, 5485-5496; Weger et al. (1997) J. Virol. 71, 8437-8447). Using this Rep 78-deficient helper construct therefore results in an increase in the expression of the smaller Rep proteins Rep 40 and Rep 52, and also of the capsid proteins, and, as a result, in the desired increase in packaging efficiency.

In general, the use of viral vectors as viral transduction vectors in gene therapy requires relatively large quantities of recombinant virus particles. As it is used within the context of this invention, the expression “recombinant” refers to a genetic unit which is altered as compared with the unit which is found naturally. Consequently, methods which can be used to achieve a high yield of recombinant virus particles are of great economic importance. Some methods are known in the prior art, for example using the herpes simplex amplicon system to optimize rAAV production (Feudner et al. (2001) J. Virol. Meth. 96:97-105), using a recombinant adenovirus, which contains rep and cap, as a helper virus (Zhang et al. (2001) Gene Ther. 8:704-712) or preparing stable AAV producer cell lines (Clark et al. (1995) Hum. Gene Ther. 6:1329-1341).

Despite these methods which are known in the prior art, the yield of recombinant virus particles is frequently still inadequate. This applies, in particular, to the cases in which cells which are stably transfected with helper genes and/or helper constructs are used for producing recombinant virus particles (see, e.g., Grimm and Kleinschmidt (1999) Hum. Gene Ther. 10:2445-2450, pages 2447-2448 “Use of producer cell lines”).

The object of the present invention is therefore to provide compositions and methods which make it possible to achieve an increase in yield when producing recombinant virus particles.

The object is achieved by using a DNA methylation inhibitor for preparing viral vectors which are derived from parvoviruses.

Surprisingly, it has emerged, within the context of the present invention, that adding methylation inhibitors when preparing viral vectors which are derived from parvoviruses can markedly increase packaging efficiency and consequently substantially increase the yield of vectors. This applies particularly to rAAV and, very particularly, to the preparation of rAAV in stable AAV packaging and/or producer cell lines. In the context of the present invention, it is possible, for example, to prepare rAAV efficiently in stable AAV packaging and/or producer cell lines. This thereby now makes it possible, for the first time, to work with stable cell lines, which are designed such that a high degree of safety exists due to recombination events being virtually ruled out, when preparing AAV on a large scale. Such an effect, which is based on a methylation or its inhibition, when preparing viral vectors derived from parvoviruses has not previously been described in the art (see, e.g., Tamayose et al. (1996) Hum. Gene Ther. 7:507-13; Inoue and Russell (1998) J. Virol. 72:7024-31; Liu et al. (1999) Gene Ther. 6:293-9; Chadeuf et al. (2000) J. Gene Med. 2:260-8; Tessier et al. (2001) J. Virol. 75:375-83).

The present invention consequently relates to the use of a DNA methylation inhibitor for preparing viral vectors which are derived from parvoviruses.

According to the invention, the expression “viral vector” relates to recombinant viruses. See above for the definition of the term “recombinant”. When the term is used for a virus, this means that the virus carries one or more nucleic acid(s) which has/have been prepared by a combination of cloning, restriction and/or ligation steps and which do(es) not occur naturally in the virus.

The following definitions are of general importance within the context of the present invention:

The terms “genes” and/or “gene sequences” refer to a polynucleotide which possesses at least one open reading frame and is able to form a particular protein by means of transcription and translation.

The term “protein” refers to an amino acid polymer of any length. The term likewise includes proteins which have gone through post-translation modification steps such as glycosylation, acetylation or phosphorylation.

Within the meaning of the present invention, the terms “nucleic acid”, “DNA” and “polynucleotide” are understood as signifying polymeric forms of nucleotides of any length, with the term only relating to the primary structure of the molecule. This term therefore encompasses both single-stranded and double-stranded DNA molecules and modified polynucleotides, such as methylated or protected (=capped) polynucleotides.

According to the invention, the expression “derived from a parvovirus” refers to a vector which contains AAV sequences or sequences of another parvovirus, with it being possible for these sequences to have been altered.

According to the invention, the expression “DNA methylation inhibitor” refers to any substance which is able to inhibit the methylation of DNA.

The use, according to the invention, of the DNA methylation inhibitor leads to an increase in packaging efficiency to a level of at least 10⁶, especially 10⁷, preferably 10⁸, in particular 10⁹, transducing particles/ml of crude lyzate or of at least 10¹⁰, especially 10¹¹, preferably 10¹², in particular 10¹³, genomic particles/ml of crude lyzate. This corresponds to 10⁵ to 10⁸ genomic particles per sown cell. The packaging efficiency can be determined indirectly by using suitable cell lines to determine the transducing AAV titer. While this was effected specifically in this present case by measuring the B7.2-positive cells by fluorescence-labeling B7.2 and counting by means of FACS analysis, another common protein detection method can be used in the case of other transgenes. A corresponding determination is shown in FIG. 1. The transducing titer depends on the detection method and on the cell type employed, which means that it is necessary to select suitable cells when choosing the system for detecting the corresponding parvovirus or AAV serotype, for example HeLa cells in the case of AAV-2. The genomic titer is independent of the cell type. The genomic titer can be ascertained by means of real-time PCR, for example as described by Veldwijk M R et al. (2002) Mol. Ther. 6:272-8.

The use according to the invention consequently increases packaging efficiency in stably transfected cells by up to 27-fold, with values of at least 5-fold, especially 10-fold, preferably 20-fold and, in particular, 25-fold also being possible in accordance with the invention. These numerical values relate to a comparison with experiments performed without using DNA methylation inhibitors.

According to a preferred embodiment of the present invention, a “DNA methylation inhibitor” refers to any arbitrary low molecular weight substances, i.e. a nucleoside analog (or nucleotide analog), peptide or antibody, or to molecules, molecular complexes or genes, which are able to inhibit the methylation of DNA or to demethylate a methylated DNA.

“Low molecular weight substances” are to be understood as being molecules, compounds and/or compositions or substance mixtures, in particular low molecular weight, organic or inorganic molecules or compounds, preferably molecules or compounds having a relative molar mass of up to approx. 1000, in particular approx 500.

According to a particularly preferred embodiment, the DNA methylation inhibitor is able to inhibit the activity of DNA cytosine methyl transferase (DNMT).

According to particularly preferred embodiments, the DNA methylation inhibitor is therefore a nucleoside analog, a low molecular weight inhibitor, a DNA-derived direct inhibitor of DNA cytosine methyl transferase, an antisense oligonucleotide inhibitor of DNA cytosine methyl transferase (see Szyf, M., Curr. Drug Targets (2000), 1:101-118) or a herpesvirus, in particular an HSV or herpesviral genes.

Examples of nucleoside analogs are 5-azacytidine or its deoxy analog 5-azadeoxycytidine or 5-fluorocytosine.

Examples of low molecular weight inhibitors are S-adenosylhomocysteine or EGX30P (EpiGen X).

Examples of DNA-based direct inhibitors of DNA cytosine methyl transferase are short phosphorothioate-modified oligonucleotides which exhibit a hairpin structure and carry a number of methylated CGs on one arm of the hairpin structure and unmethylated CGs on the other arm, thereby resembling the hemimethylated substrate of DNA cytosine methyl transferase (Szyf, M., see above).

Examples of antisense oligonucleotide inhibitors of DNA cytosine methyl transferase are specific antisense oligonucleotides which have been investigated (screened) and selected both for the mouse DNMT1 mRNA and the human DNMT1 mRNA (Ramchamdani, S. et al. (1997) Proc. Natl. Acad. Sci. USA 94:684-689; Fournel, M. et al. (1999) J. Biol. Chem. 274:24250-24256).

According to a very particularly preferred embodiment, the DNA methylation inhibitor is therefore selected from the group comprising 5-azacytidine, 5-azadeoxycytidine, 5-fluorocytosine, S-adenosylhomocysteine and EGX30P.

According to another very particularly preferred embodiment, the DNA methylation inhibitor is a herpesvirus, preferably an HSV, in particular an HSV-1 or HSV-2, or viruses which are derived therefrom. Without being committed to one mechanism, infection with a herpesvirus appears to alter cellular gene expression such that this altered expression leads to a decrease in the methylation of genes. Other conceivable mechanisms are that herpesviruses themselves encode an enzyme which reverses the methylation of genes or else that herpesviruses are able to bring about the transcription of genes regardless of their methylation status.

Herpesviral genes represent another very particularly preferred embodiment of a DNA methylation inhibitor. The term “herpesviral genes” is understood as meaning the genes of herpesviruses whose gene products inhibit or abolish the methylation of DNA or make transcription methylation-independent. Thus, instead of infecting the cells with a herpesvirus for the purpose of producing the AAV, the herpesviral genes can also be transfected transiently, or in particular stably, into the cells and thus function as methylation inhibitors in analogy with an infection with a herpesvirus.

According to a preferred embodiment, the viral vector is, within the context of the use according to the invention, produced in a cell which contains the genes which are required for forming virus particles. In this regard, the reader is referred to the above remarks concerning genes which are required for forming the particles. The cells which are used are eukaryotic cells, preferably mammalian cells, particularly preferably insect cells or human cells or cell lines, in particular HeLa cells, A549 cells, K209 cells, B50 cells or Z211 cells (for the latter, see Gao G. et al. (2002) Mol. Ther. 5:644-649). In principle, it is possible to use any cell or cell line which is permissive for, i.e. accessible to, the vector construct, the helper construct and, where appropriate, the helper virus.

According to a preferred embodiment, and within the context of the use according to the invention, the viral vector is prepared from a vector cell. The term “vector cell” refers to a cell which contains at least one vector construct but no helper construct. This means that the vector cell constitutes the starting material for preparing the viral vectors and that additional factors which are important for the particle formation, such as helper constructs, are added during the preparation process.

According to a preferred embodiment, and within the context of the use according to the invention, the viral vector is prepared from a packaging cell. The term “packaging cell” refers to a cell which contains at least one helper construct but no vector construct. This means that the packaging cell constitutes the starting material for preparing the viral vectors and that additional factors which are important for the particle formation, such as vector constructs, are added during the preparation process.

According to a preferred embodiment, and within the context of the use according to the invention, the viral vector is prepared from a producer cell. The term “producer cell” refers to a cell which contains both at least one helper construct and at least one vector construct. This means that the producer cell constitutes the starting material for preparing the viral vectors. If helper genes are required for preparing the viral vectors, they are added during the preparation process.

According to another preferred embodiment, the packaging cell, the vector cell and/or the producer cell is/are stably transfected with helper constructs. The skilled person is familiar with methods for transfecting stably (see, e.g., Gao et al. (2002) Mol. Ther. 5:644-649).

According to a particularly preferred embodiment, the individual genes are present on the helper construct(s), in the packaging and producer cell lines, in a functionally separate form (for definition, see above).

Therefore, in a preferred embodiment, not more than 50%, preferably not more than 20%, particularly preferably not more than 10%, of the inethylation sites which are present are methylated in the viral vector which is prepared within the context of the use according to the invention.

Surprisingly, it has been found, in the present invention, that, when viral vectors are being prepared, it is, in particular, the methylation of the rep and cap genes, or even only of their promoters, which is responsible for the low yield of rAAV particles. It was possible to demonstrate this by the fact that an additional transfection of the corresponding plasmid(s) containing rep and cap genes also restored the production of rAAV in 11-20-23 cells which were infected with adenoviruses. 11-20-23 cells were produced by stably transducing (with rAAV-(B7.2/GM-CSF)) a stable packaging cell line (stable expression of Rep and Cap). The packaging cell line is a subclone of the cell line C97 (see Examples 8 and 9), which is described in WO 02/20748 and which is based on HeLa cells. The restoration of the rAAV production by an additional transfection of the corresponding plasmid(s) containing rep and cap genes means that the methylation of cellular genes or their promoters, which methylation must have continued to persist in the described case, evidently has no negative influence on the preparation of the viral vectors. This experiment is a further indication that parvoviruses cannot be compared with other virus families since these latter, in contrast to parvoviruses, do not possess any rep or cap genes. Without being bound to a theory, it is consequently probable that methylation of the rep and cap genes, which are involved in producing viral vectors, is a reason for the low yield which is frequently observed when preparing viral vectors.

For this reason, in another preferred embodiment of the invention, the DNA methylation inhibitor is used to inhibit the methylation of the methylation sites which are present in the rep and cap genes or their promoters. In this connection, the methylation inhibition relates to any form of the rep and cap genes as are used in the present invention, for example in packaging cell lines in which the rep and cap genes are present in functionally separate form and transfected episomally or integrated stably into the host cell genome.

According to another preferred embodiment, the viral vector which is derived from parvoviruses contains a vector construct.

As explained above, it is necessary in the case of a number of viral vectors, such as AAV, for helper genes, which are derived from other viruses, i.e. what are termed helper viruses, also to be present for achieving particle formation. In this case, the invention first of all includes these helper genes being present in the cell in which the viral vector is prepared. The helper genes can, for example, be stably integrated or be present episomally.

However, according to a preferred embodiment of the invention, a helper virus which contains the corresponding helper genes in its genome is additionally added for the purpose of preparing the viral vector. Examples of suitable helper viruses are known to the skilled person and include adenoviruses, herpes simplex viruses and vaccinia viruses.

In principle, the DNA methylation inhibitor can be added at any stage in the production of the viral vectors up to the time of harvesting the viral vectors. However, it is preferably added at the time of, or prior to, activating the virus production, for example by means of infecting with a helper virus in the case of AAV production, or otherwise activating virus production.

It has turned out that it is particularly advantageous to use the methylation inhibitor as early as possible. According to a particularly preferred embodiment, the DNA methylation inhibitor is used prior to adding the helper virus, if the addition of a helper virus is required for producing viral particles.

According to a preferred embodiment, the viral vector which is derived from a parvovirus is derived from an adenoassociated virus (AAV) (Pfeifer and Verma (2001) Annu. Rev. Genomics Hum. Genet. 2:177-211).

According to a particularly preferred embodiment, the viral vector is derived from AAV-1, AAV-2, AAV-3, AAV-4, AAV-5, AAV-6, AAV-7 or AAV-8.

Examples of these viral vectors are well known (see above).

According to a preferred embodiment, a vector construct, which comprises the transgene(s), which is flanked by one, preferably two ITR regions, and one or more helper construct(s), which comprise(s) the rep and cap genes, are used for producing rAAV. For more precise explanation, the reader is referred to the above passages which describe the production of AAV vectors. According to a particularly preferred embodiment, use is additionally made of a helper virus, for example adenovirus, herpesviruses or vaccinia viruses.

According to a preferred embodiment, HeLa cells are used for producing viral, AAV-derived vectors. However, it is in principle possible to use any cell line which is permissive for, i.e. accessible to, the vector construct, the helper construct and, where appropriate, the helper virus.

According to a preferred embodiment, a packaging cell line, which contains copies of the entire AAV genome, without the flanking ITRs and with rep and cap genes under the control of their natural viral promoters (see above), is used for producing viral, AAV derived vectors. These promoters, i.e. P5, P19 and P40, are inactive in the absence of an infection with a helper virus (Inoue and Russell (1998) J. Virol. 72:7024-7031; Gao et al. (1998) Human Gene Therapy 9:2353-2362).

According to a particularly preferred embodiment, the packaging cell line is transfected with a vector construct.

After that, and according to another preferred embodiment, the cell line which is transfected with the vector construct is transduced with a helper virus, with this inducing expression of the AAV genes. Such cell lines can be used for producing large quantities of AAV, especially for commercial applications (see above).

According to a very particularly preferred embodiment, a producer cell line (for definition, see above) is used instead of a packaging cell line. This means that it is not necessary to transfect with a vector construct. On the other hand, transducing with a helper virus is necessary as a rule.

According to a very particularly preferred embodiment, the rep and cap genes are functionally separated (see above and DE 10044348) in the packaging or producer cells.

According to a preferred embodiment, the packaging cell, vector cell or producer cell is helper virus-independent, i.e. producing the AAV does not require any infection with a helper virus. Such a helper virus-independent packaging cell, vector cell or producer cell normally contains genes which are required for inducing AAV production under the control of an inducible promoter. These genes can be of viral or cellular origin.

According to a particularly preferred embodiment, the cap gene in the packaging or producer cells is also under the control of the other homologous promoters which are actually assigned to the rep gene.

According to a very particularly preferred embodiment, these promoters are P5, P19 and P40 in the case of AAV-2 (see above and DE 10044348).

According to a very particularly preferred embodiment, the expression “functionally separate” means, in this context, that the rep gene and the cap gene do not overlap even in regard to their promoters. This is achieved, for example, by both parts of the jointly used coding sequence and the P40 promoter being duplicated (see, for example FIG. 8). According to the invention, this can mean that the genes can be arranged in various ways in a genome. In the first place, the genes can be located at different sites in the cell, for example be integrated at different sites in the genome, be present on plasmids and also partly integrated and be present on plasmids. In the second place, the genes can also be located alongside each other on the same DNA molecule, for example a chromosome or a plasmid, with each gene being controlled by its promoter. Such an arrangement is probable, for example, when two genes are transfected together on different DNA molecules. These molecules can form concatamers during the transfection with these concatamers then integrating into the genome at one site but still forming functionally independent units.

According to preferred embodiments, the functionally separate rep and cap genes are present in the packaging or producer cells in transiently transfected, that is episomal, form or are integrated into the cellular genome at the same site, for example as concatamers, or at different sites. The advantage of such an arrangement is that in each case at least two independent recombination events, which, at a frequency of in each case 10⁻⁷ per cell division, that is 10⁻¹⁴ in total, are extremely rare, would be required for reconstituting replication-competent wild-type AAV (rcAAV) particles. In fact, it was not possible to detect any rcAAV in a recombinant virus preparation which contained 2×10¹⁰ genomic particles.

According to another preferred embodiment, the P5, P19 and P40 promoters in the cap gene are altered by mutagenesis such that while the promoter function is intact in regard to the start of transcription, these constructs are unable to express any functional Rep protein (see above). The skilled person is familiar with other possibilities of using mutagenesis for inactivating expression of the Rep protein, e.g. by means of inactivating the transcription start.

According to a very particularly preferred embodiment, the two large Rep proteins Rep 68 and Rep 78 are provided, for the adenovirus-inducible transactivation of the P40 promoter, from a second source (in trans or in cis, for definition, see above). It was now possible to stably integrate these Rep expression constructs into the genome of the host cells. These constructs have the advantage that, while no toxic quantities of Rep proteins are expressed in the absence of a helper virus, the constructs ensure very strong, helper virus-inducible expression of Rep protein.

According to another preferred embodiment, one type of preferred helper construct for packaging rAAV contains nucleic acid sequences which encode at least one Rep protein, with, in the case of AAV-2, for example, Rep proteins being understood as being the proteins Rep 78, Rep 68, Rep 52 and Rep 40, in particular Rep 68, Rep 52 and Rep 40, especially Rep 68 and Rep 52. The other type of preferred helper construct contains nucleic acid sequences which encode at least one of the known Cap proteins, with the Cap proteins being the proteins VP1, VP2 and VP3. The genes for these proteins, and also the ITR sequences, can be isolated from wild-type AAV, which can be obtained generally in the form of clones. Thus, for example, the clone pSM620 is described in Samulski et al. (1982) Proc. Natl. Acad. Sci. USA 79:2077, while the clone pAV1 is described in Laughlen et al. (1983) Gene 23:65 and the clone sub201 is described in Samulski (1987) J. Virol. 61:3096.

According to a particularly preferred embodiment, expression of the Rep proteins is, in the case of AAV-2, controlled by the natural promoters P5 and P19 (P5 for Rep 78/Rep 68 and P19 for Rep 52/Rep 40), while expression of the Cap protein is controlled by the natural AAV promoter P40; in particular by the natural AAV promoters P19 and P40, especially by the natural AAV promoters P5, P19 and P40. The same applies, in a corresponding manner, to the complementary promoters of the other AAV serotypes.

According to a particularly preferred embodiment, the Cap expression plasmid for AAV-2 contains the AAV promoters P5, P19 and P40 in order to permit expression which is regulated in dependence both on helper virus infection or helper virus gene products and on Rep protein expression, since this arrangement best reflects the natural lytic AAV life cycle. Using the natural AAV regulatory sequences ensures that transcription factors which are required for the regulated expression of the cap genes find all the binding sites in the natural promoter region for the purpose of exerting their regulatory functions (see above).

According to another preferred embodiment, expression of the Rep protein and expression of the Cap protein in the cell are regulated independently of each other. This approach was chosen because it was found that weak expression of Cap is initially required for efficiently packaging rAAV in stable cell lines since, otherwise, high quantities of Cap would have a toxic effect on cells and/or large quantities of empty capsides would be formed. On the other hand, Cap must be expressed strongly at the time of packaging. A constitutive, heterologous promoter is unable to fulfill both these criteria simultaneously. While this can be improved by using inducible, heterologous promoters, it is in practice extremely difficult to use these promoters to implement the precise chronological regulation and the strength of the Cap expression. Using the natural homologous promoters couples the expression of Cap to the activation by helper virus gene products and/or cellular helper genes, as well as Rep, and consequently regulates it chronologically exactly as in the wild-type situation.

According to another preferred embodiment, the transcription of the nucleic acids encoding the Rep proteins and the Cap proteins is terminated by the natural regulatory sequences, in particular by the natural AAV polyA signal. As when initiating transcription, the use of homologous sequences for terminating transcription of the AAV cap and rep genes also increases the quantity of the rAAV particles produced by the AAV vector system.

Expediently, the cell which is employed, within the context of the use according to the invention, when producing viral vectors in general and AAV vectors in particular is a eukaryotic cell, preferably a mammalian cell, particularly preferably an insect cell or human cell or cell line, in particular HeLa cells, A549 cells, K209 cells, B50 cells or Z211 cells (for the latter, see Gao G. et al. (2002) Mol. Ther. 5:644-649). In principle, it is possible to use any cell or cell line which is permissive for, i.e. accessble to, the vector construct, the helper construct and, where appropriate, the helper virus. HeLa cells have proved to-be particularly advantageous since the AAV P5 promoter is virtually inactive in HeLa cells and it is therefore possible to stably, integrate a cassette for expressing the AAV Rep protein under the control of the natural regulatory elements into their genome such that the Rep protein does not have a toxic effect in these cells (Clarke et al. (1995) Human Gene Therapy 6, 1229-1341; Tamayose et al. (1996) Human Gene Therapy 7, 507-513; Inoue & Russell (1998) see above; Gao et al. (1998) see above).

According to a particularly preferred embodiment, the helper construct contains nucleic acid sequences which encode at least one Rep protein, with the Rep proteins being Rep 68, Rep 52 and/or Rep 40 but not Rep 78. The advantage of these Rep 78-deficient helper constructs is that the largest Rep protein, which is most toxic for the packaging cells and which has the greatest inhibitory activity on cellular processes such as transcription and the cell cycle, is not expressed at all. The packaging efficiency can therefore be increased when using this helper construct,- due to the absence of Rep 78. Both Rep 68 and Rep 78 are expressed by the P5 promoter in the natural system. Using the Rep 78-deficient helper construct is furthermore advantageous because Rep 68 is a stronger transactivator than Rep 78 of the AAV promoters P19 and P40 in adenovirus-infected cells (Hörer et al. (1995) J. Virol. 69, 5485-5496; Weger et al. (1997) J. Virol. 71, 8437-8447). Using this Rep 78-deficient helper construct therefore results in an increase in the expression of the smaller Rep proteins Rep 40 and Rep 52, as well as of the capsid proteins, and consequently in the sought-after higher packaging efficiency.

For the purpose of preparing the Rep 78-deficient helper construct pUCRep68,52,40Cap(RBS)dl37 (cf. FIG. 9), the AAV sequences from nucleotide 201 to nucleotide 4497, including the deletion of the intron sequence, and also from nucleotide 658 to nucleotide 4460 were cloned into the bacterial expression plasmid pUC19, with the binding sites for the Rep protein in the pUC19 sequence being deleted (cf. DE 19905501, Example 5). As a result of using this strategy, two rep genes and at least two cap genes, in each case possessing its own poly(A) sequence for terminating transcription, are arranged one behind the other. The Rep proteins Rep 68 and Rep 40, and also the Cap proteins VP2 and VP3, can be expressed from the first segment (AAV sequence, nucleotide 201 to nucleotide 4497) whereas the Rep proteins Rep 52 and Rep 40, and also the Cap proteins VP1, VP2 and VP3, can be expressed from the second segment (AAV sequence nucleotide 658 to nucleotide 4460). Taken overall, this thereby encodes all the AAV-2 proteins with the exception of Rep 78.

For the purpose of preparing the helper construct pUCdlRep78Cap(RBS)dl37 (cf. FIG. 7), which is likewise Rep 78-deficient, said AAV sequences (nt 201-2310; nt 658-4460, including the deletion of the intron sequence) were likewise cloned into the bacterial expression plasmid pUC19 (cf. DE 19905501, Example 5). In this connection, the binding sites for the Rep protein in the pUC19 sequence were once again deleted. In this way, the rep gene was partially duplicated. The resulting helper construct contains only one poly(A) sequence, which means that all mRNA transcripts possess the same 3′ end. The Rep proteins Rep 68 and Rep 40 can be expressed from the first segment (AAV sequence, nucleotide 201 to nucleotide 2310), while the Rep proteins Rep 52 and Rep 40, and also the Cap proteins VP1, VP2 and VP3, can be expressed from the second segment (AAV sequence, nucleotide 658 to nucleotide 4460). Taken overall, therefore, this vector construct also encodes all the AAV-2 proteins with the exception of Rep 78.

For the purpose of preparing a helper construct pUCdlRep78dlCap(RBS)dl37 for expressing the Rep proteins Rep 68, Rep 52 and Rep 40, the AAV nucleotides 2945 to 4046 were deleted from the cap gene (nucleotides 2203 to 4410) of the helper construct pUCdlRep78Cap(RBS)dl37. As a result of this deletion, it is no longer possible to express any functional Cap proteins.

In principle, the reader is referred, for the purpose of elucidating the preferred embodiments, to the passages above in which the preparation of AAV is explained generally.

According to a preferred embodiment of the invention, the vector constructs for AAV vectors contain one or more nucleic acids which are heterologous to AAV and which are flanked by one, preferably two ITR sequences, with the ITR sequence which is located 5′ possessing a deletion in the region of the C palindrome.

According to a preferred embodiment, the deletion within the 5′-flanking ITR sequence comprises 80 nucleotides, in particular 40 nucleotides, especially 22 nucleotides, in the region from nucleotide 61 to nucleotide 82.

According to a very particularly preferred embodiment, these vector constructs contain the AAV sequences 1-60/83-191 (ΔC-Arm ITR as the left ITR—see DE 10044384) and 4498 to 4671 (as the right ITR).

These vector constructs contain, for example, one or more nucleic acids which are heterologous to AAV, in particular a nucleic acid encoding a protein selected from a cytokine, in particular IL2, IL4, IL12 and/or GM-CSF (granulocyte macrophage colony-stimulating factor) and/or a costimulatory molecule, in particular B7, especially B7.1 and/or B7.2. However, according to the present invention, any arbitrary coding or noncoding nucleic acid sequence can be used as a heterologous nucleic acid sequence. Preferably, one or more heterologuos nucleic acid sequence(s) is/are introduced into a replication-deficient vector construct using conventional cloning techniques which are known to the skilled person (Sambrook et al. (1989) Molecular Cloning: A Laboratory Manual, Cold Spring Harbor Laboratory, Cold Spring Harbor, N.Y.).

Other suitable nucleic acid sequences are sequences encoding chemokines such as lymphotactin, RANTES, MCP-1 or Mip-1α, cytokines such as IL12, IL7, IL18, IL2, GM-CSF, IL1, IL6, interferon γ or IL10, or antibodies, antibody fragments or single-stranded antibodies, for example directed against ICOS and, in addition, against the ICOS receptor, CD40, CD40 ligands, VEGF, IL1 and TNF-α, against tumor antigens such as Her-2/neu, GD3 or CA125, against viral antigens or against IgE; and, in addition, against soluble receptor forms such as ICOS FC, ICOS ligand FC, CD40L FC or TNF-α receptor FC, or against apoptosis-inducing molecules such as proteins of the BCL-X family, BAX, BAD or caspases, necrosis-inducing peptides such as perforins, toxins, for example derived from bacteria, viral tumor antigens such as HPV E6 or E7, or nonviral tumor antigens such as B lymphoma-specific idiotype antibodies, BCRA-1, CA 125, α-fetoprotein, CEA and p53. Sequences encoding blood coagulation factors, such as factor VIII or factor IX, or factors from other known monogenetic hereditary diseases, are also suitable.

According to another preferred embodiment, the vector construct contains sequences which do not encode polypeptides, for example for use as ribozymes, antisense RNAs or interfering RNAs (RNAᵢ), which sequences are consequently also encompassed by the term “transgene”.

Accordingly, within the context of the use according to the invention, use is made of a cell, in a particularly preferred embodiment for producing rAAV, which contains at least one copy of a helper construct for expressing at least one AAV Rep protein and at least one AAV Cap protein and, in addition, at least one copy of a recombinant vector construct. The vector construct is in turn characterized by the fact that it carries a foreign DNA which is flanked by at least one ITR region.

According to a particularly preferred embodiment, the nucleic acids which encode the Rep protein and the Cap protein are functionally separated and are operatively linked to the natural AAV regulatory sequences.

When a herpesvirus is used for producing a viral vector in cell lines which are stably transfected with helper constructs, the packaging efficiency is increased, according to the invention, to a level of at least 10⁹, especially 10¹⁰, preferably 10¹¹ or 10¹², in particular 10¹³, genomic particles/ml of crude lyzate, corresponding to 10⁵ to 10⁸ genomic particles per sown cell. This means an up to 26-fold increase in packaging efficiency in the case of stably transfected cells.

The term “herpesviral genes” is understood as meaning the genes of herpesviruses whose gene products inhibit or abolish the methylation of DNA or make transcription methylation-independent (see above).

The invention consequently also relates to the use of a herpesvirus or of herpesviral genes for producing a viral, parvovirus-derived vector, in cell lines which are stably transfected with helper constructs, for the purpose of increasing packaging efficiency at least 5-fold, especially 10-fold, preferably 20-fold and, in particular, 25-fold as compared with producing the vector while using an adenovirus without any addition of DNA methylation inhibitors.

The above remarks and embodiments in relation to packaging cells, vector cells, producer cells, helper constructs, vector constructs and viral vectors relate in exactly the same way to this part of the subject matter of the invention.

It was surprisingly also possible, within the context of this invention, to abolish the observed inhibition of the packaging efficiency, when using packaging and producer cell lines and AdV as the helper virus, by solely employing viruses of the herpes family, such as HSV-1, as the helper virus. This applies, in particular, to AAV and was not to be expected from previous descriptions in regard to the general use of HSV-1 as a helper virus for AAV (e.g. Weindler and Heilbronn (1991) J. Virol. 65:2476-83; Conway et al. (1997) J. Virol. 71:8780-9).

The advantage of this part of the subject matter of the present invention is that using herpesviruses as helper viruses when producing viral vectors can markedly increase the packaging efficiency. This applies, in particular, to rAAV and, very particularly, to the production of rAAV in stable AAV packaging and/or producer cell lines. Consequently, it has been demonstrated that rAAV can be produced efficiently in stable AAV packaging and/or producer cell lines.

According to this invention, the expression “herpesvirus” refers to any virus of the herpesvirus family, whether naturally occurring or recombinant. Examples of naturally occurring herpesviruses are HSV-1, HSV-2, HSV-3 (varicella zoster), HSV-4 (Epstein-Barr virus), HSV-5 (cytomegalovirus), HSV-6, HSV-7 and HSV-8, or animal herpesviruses. Recombinant herpesviruses are disclosed in the prior art. Oncolytic herpesviruses, such as G207 or NV1020, are suitable, for example.

According to a preferred embodiment, the viral vector is derived from a parvovirus, particularly preferably from an AAV, in particular from AAV-1, AAV-2, AAV-3, AAV-4, AAV-5, AAV-6, AAV-7 or AAV-8.

Since a problem of producing AAV while making use of induction with helper viruses is that of subsequently inactivating the helper viruses whereas the AAV which have been produced have to remain active, a preferred embodiment of this invention is that of employing recombinant herpesviruses. This embodiment is not restricted to AAV but applies generally to all uses within the context of the present invention. The literature discloses many replicating or nonreplicating herpesviruses, e.g. G207 or NV1020. These viruses have the advantage that it has been shown, in human clinical studies, that such recombinant herpesviruses are safe and can be injected into humans. For this reason, the use of such recombinant viruses contributes to the safety of an AAV preparation since slight contaminations of AAV products are not dangerous to humans.

In addition, another very particularly preferred embodiment is the use of herpesviral helper genes for producing a viral, parvovirus-derived vector, in cell lines which are stably transfected with helper constructs, for purpose of increasing packaging efficiency. The term “herpesviral helper genes” is understood as meaning the genes of herpesviruses whose gene products are necessary for, or promote, replication of the AAV (see above). Thus, instead of infecting the cells, for the AAV preparation, with a herpesvirus, it is also possible to transiently or, in particular, stably transfect the herpesviral helper genes into the cells. Appropriate helper genes are described above. Included. These can, for example be the seven replication genes UL5, UL8, UL9, UL29, UL30, UL42 and UL52. UL 5, 8 and 52 form the HSV helicase primase complex, UL29 encodes the single-stranded DNA-binding protein, UL42 encodes a double-stranded DNA-binding protein, UL30 encodes the HSV DNA polymerase and, finally, UL9 encodes a protein which binds the HSV origin of replication (see Weindler F W and Heilbronn R (1991) J. Virol. 65(5):2476-83).

The invention furthermore relates to a process for producing viral, parvovirus-derived vectors, which comprises using a DNA methylation inhibitor during the production.

Preferred embodiments of the process according to the invention with regard to DNA methylation inhibitors, packaging cells, vector cells, producer cells, helper constructs, vector constructs and viral vectors correspond to those of the above use according to the invention.

Within the context of the use according to the invention, or of the process according to the invention, a slightly methylated or unmethylated viral vector is produced. In particular, slight methylation, or no methylation, of the rep and cap genes and/or their promoters is preferably required for the increase in the yield of viral, parvovirus-derived vectors.

According to another preferred embodiment, the viral, parvovirus-derived vector contains a vector construct.

The present invention therefore also relates to a vector construct in which not more than 50%, preferably not more than 20%, particularly preferably not more than 10%, of the methylation sites which are present are methylated.

An advantage of such vector constructs, which are only slightly methylated or unmethylated, is that, because of the low degree of methylation, these constructs are more strongly expressed than comparable methylated genes when transduced into the recipient cells, particularly mammalian cells, very particularly human cells.

Consequently, the present invention provides processes and uses which make it possible to increase the yield when producing viral vectors.

The invention is clarified below by means of Examples and Figures.

### BRIEF DESCRIPTION OF THE FIGURES

FIG. 1 shows the titer (transducing particles (tp) per ml) of a transducing rAAV-(B7.2/GM-CSF) (based on AAV-2) three days after HSV-1 infection or after AdV infection (in each case, MOI 10). AdV was administered either alone or in combination with 50 mM Na butyrate (But), 3 μM trichostatin A (TSA) or 3 μM 5-azacytidine (Aza) (Sigma, Deisenhofen). “Standard” means transient cotransfection of HeLa cells with pAAV-(B7.2/GM-CSF) and the AAV-2 rep/cap helper plasmid and infection with adenovirus (AdV) (MOI 10); harvesting, 3 days after infection;

FIG. 2 shows a Western blot analysis of the expression of AAV-2 Rep and Cap in producer cells, in each case after infection with AdV or HSV-1 (in each case MOI 10).

FIG. 3 shows a diagram of the vector construct pAAV-(B7.2/GM-CSF).

FIG. 4 shows the diagram of the helper construct pUCp5Repdl37, which encodes Rep40, Rep52, Rep68 and Rep78.

FIG. 5 shows the diagram of the helper construct pUCp5p19p40Capdl37, which encodes VP1, VP2 and VP3.

FIG. 6 shows the diagram of the helper construct pUCdlRep78dlCap(RBS)dl37, which encodes Rep40, Rep52 and Rep68.

FIG. 7 shows the diagram of the helper construct pUCdlRep78Cap(RBS)dl37, which encodes Rep40, Rep52 and Rep68 and also VP1, VP2 and VP3.

FIG. 8 shows a diagram of two helper constructs as compared with wild-type AAV. The natural AAV promoters P5, P19 and P40 are also shown, as is the major intron of the AAV genome (“I”) and the natural poly(A) signal of the AAV genome (“pA”). The coding sequences of the AAV Rep and Cap genes are also shown.

FIG. 9 shows a diagram of a Rep 78-deficient helper plasmid designated pUCRep68,52,40Cap(RBS)dl37, which encodes Rep40, Rep52 and Rep68 and also VP1, VP2 and VP3.

### EXAMPLES

### Example 1

### Production of AAV in Producer Cells Using Various Inhibitors

A. Cell Culture

Revitalizing HeLa t Cells and Derivatives

A cryotube of HeLa t cells or of HeLa t-derived AAV packaging or producer cells (5×10⁶ to 1×10⁷ cells per cryotube per ml) was thawed at 37° C. in a waterbath. 10 ml of DMEM (Dulbecco's modified Eagle medium) were immediately added to the cells, which were then centrifuged at 200 g for 5 minutes. The cell pellet was resuspended in 10 ml of DMEM and the cells were centrifuged once again at 200 g for 5 minutes. The cells were then resuspended in from 20 to 30 ml of DMEM/10% FCS (fetal calf serum) and cultured at 37° C. and 5% CO₂.

Cell Culture and Transfections

HeLa t cells and the derived packaging and producer cells were kept as adherent cultures in DMEM/10% FCS at 37° C. and 5% CO₂. Neomycin was added to a final concentration of 800 μg/ml in order to select stable packaging cell clones.

The transfections were carried out using conventional calcium phosphate precipitation methods and endotoxin-free plasmid DNA, which had been prepared using Qiagen (Hilden, Germany) kits.

B. Vectors

Plasmid Construction

The plasmids (vector plasmids and helper plasmids) which were used in the context of the present invention were prepared employing standard cloning techniques as described in Sambrook et al., (1989), see above.

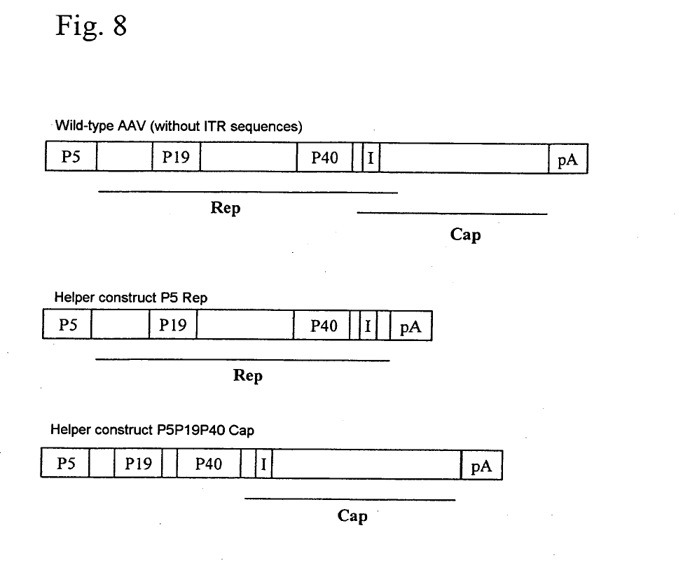

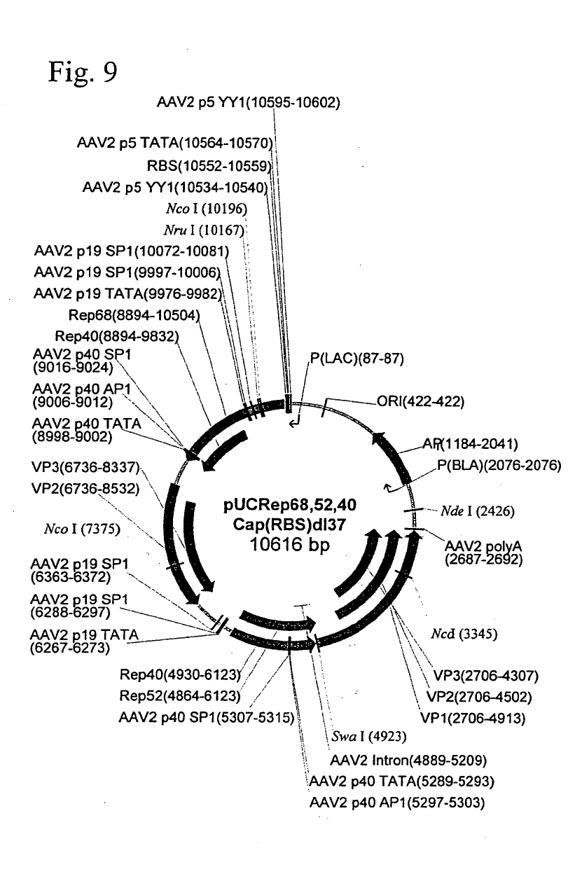

The plasmid pUCp5Rep (FIG. 8) was prepared by deleting a DNA fragment which contained nucleotides 2300-4170 of the AAV genome. (Ruffing et al. (1994) J. Gen. Virol. 75, 3385-3392 (Gene Bank Accession No. AF 043303). pUCp5Repdl37 was obtained by deleting AAV bases 4461-4497 from pUCp5Rep. The plasmid pUCp5p19p40Cap (FIG. 8) was obtained by deleting the DNA segment between nucleotides 350 to 650 and 1045 to 1700 of the AAV genome. pUCp5p19p40Capdl37 was obtained by deleting AAV bases 4461-4497 from pUCp5p19p40Cap.

The vector constructs for pAAV-(B7.2/GM-CSF) were constructed using the Promega (Germany) pCI plasmid and then transferred into a pUC19-based plasmid which contained the ITR sequences (cf. WO 00/47757).

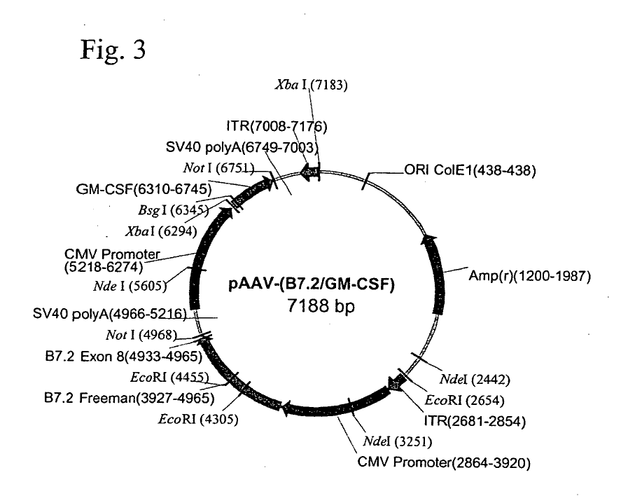

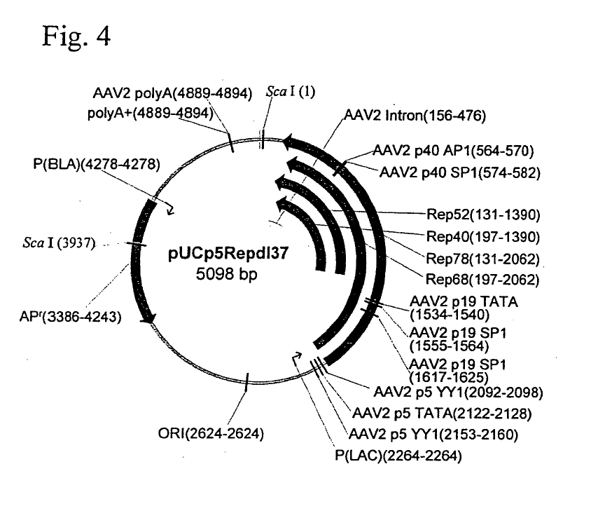

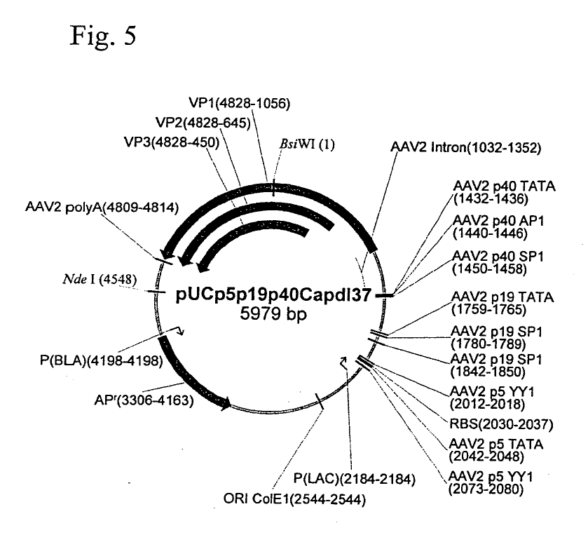

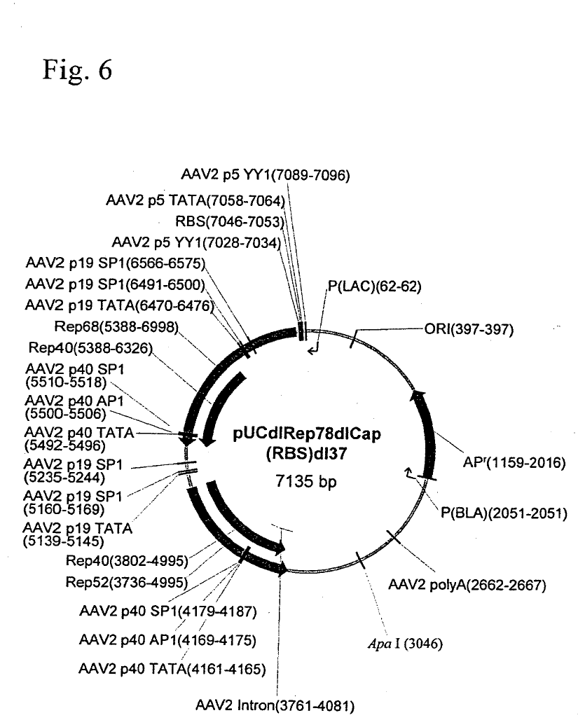

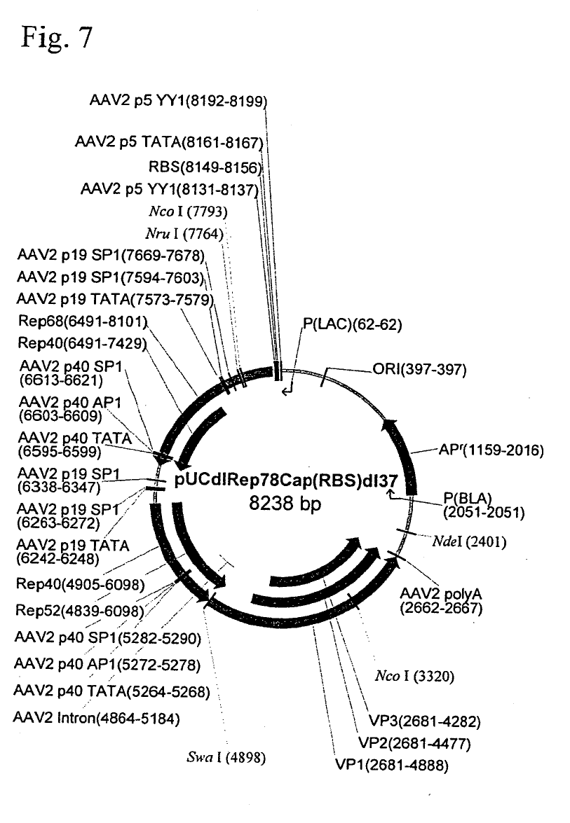

The Rep helper construct pUCdlRep78dlCapdl37 was prepared by deleting nucleotides 3046 to 4149 from the helper construct pUCdlRep78Cap (RBS)dl37; cloning, see WO 00/47757 p.26 1.14 to p.29 1.5 as pUC“ΔRep78Cap”(RBS)Δ37) using the restriction enzyme ApaI. The corresponding plasmid maps are depicted in FIGS. 3 to 7.

The helper plasmid pUCp5Repdl37 leads to the expression of all four AAV Rep proteins, i.e. Rep 78, Rep 68, Rep 52 and Rep 40. The helper plasmid pUCdlRep78dlCap(RBS)dl37 leads to the expression of Rep 68, Rep 52 and Rep 40. The helper plasmid pUCp5p19p40Capdl37 leads to the expression of all three AAV capsid proteins, i.e. VP1, VP2 and VP3. The vector plasmid pAAV-(B7.2/GM-CSF) leads to the packaging of the rAAV-(B7.2/GM-CSF) genome in AAV particles and to the expression of B7.2 and GM-CSF in the cells which are infected with these AAV particles.

C. Preparing Cell Lines

Preparing Stable Cell Lines for Packaging rAAV

HeLa t cells were cotransfected with the plasmids pUCp5Repdl37, pUCp5p19p40Capdl37 and pCI-neo (Promega) in a ratio of 10:10:1 or with pUCdlRep78dlCapdl37, pUCp5p19p40Capdl37 and pCI-neo in a ratio of 10:10:1. Stably transfected cell clones were initially selected for neomycin resistance. They were then selected for rAAV packaging efficiency by the clones being in each case transiently transfected with a vector plasmid (e.g. pAAV-(B7.2/GM-CSF, see FIG. 3) and superinfected with adenovirus. The prepared lyzates were analyzed with regard to the transducing rAAV titer. The cell clones which gave rise to the highest rAAV titers, and also gave rAAV titers which still remain the same even at a high passage number, were expanded.

Preparing a Stable Cell Line for Producing rAAV

In order to prepare a stable cell line for producing rAAV-(B7.2/GM-CSF), the above-described C97 cell line was infected stably with the recombinant virus rAAV-(B7.2/GM-CSF). Positive cell clones were selected in rAAV production tests by infection with adenovirus, analyzing the corresponding lyzates with regard to the transducing rAAV titer and expanding the clones which gave rise to the highest rAAV titers and gave rAAV titers which still remained the same even at a high passage number.

D. Packaging/Producing rAAV

Producing rAAV Using Packaging Cell Lines

rAAV was initially produced using packaging cell lines (e.g. 2.5×10⁵ cells sown in a 6 cm diameter cell culture dish, with the cells being sown 1 day before the transfection) by transiently transfecting a vector plasmid (e.g. pAAV-(B7.2/GM-CSF)) and then infecting with adenovirus (multiplicity of infection, MOI 10) 24 hours after said transfection. After the rep and cap genes which were integrated in the packaging cells had been observed to shut down, the preparation was modified in that 5-azacytidine (Sigma, Deisenhofen) was added to the cells, together with the adenovirus infection, to a final concentration of 3 μM, or the cells were only infected with HSV-1 (MOI 10) in place of the adenovirus infection.

After a further 72 hours, the lyzates were prepared by freeze/thaw lysis and freed from the cell debris. Adenovirus or HSV-1 was heat-inactivated at 56° C.

Preparing rAAV Using Producer Cell Lines

rAAV-(B7.2/GM-CSF) was initially prepared using producer cell lines (e.g. 1×10⁶ cells sown in a 6 cm diameter cell culture dish, with the cells being sown 1 day before the infection) by infecting with adenovirus (multiplicity of infection, MOI 10). After the rep and cap genes which were integrated in the producer cell lines had been observed to shut down, the preparation was modified in that 5-azacytidine (Sigma, Deisenhofen) was added to the cells, together with the adenovirus infection, to a final concentration of 3 μM or in that the cells were infected with HSV-1 (MOI 10) in place of the adenovirus infection. After 72 hours, the lyzates were prepared by freeze/thaw lysis and freed from the cell debris. Adenovirus of HSV-1 was heat-inactivated at 56° C. When rAAV are prepared using producer cell lines, the transfection step is dispensed with, which means that adherent culture of the cells is no longer required.

### Example 2

### Producing AAV in Producer Cells Using Different Inhibitors

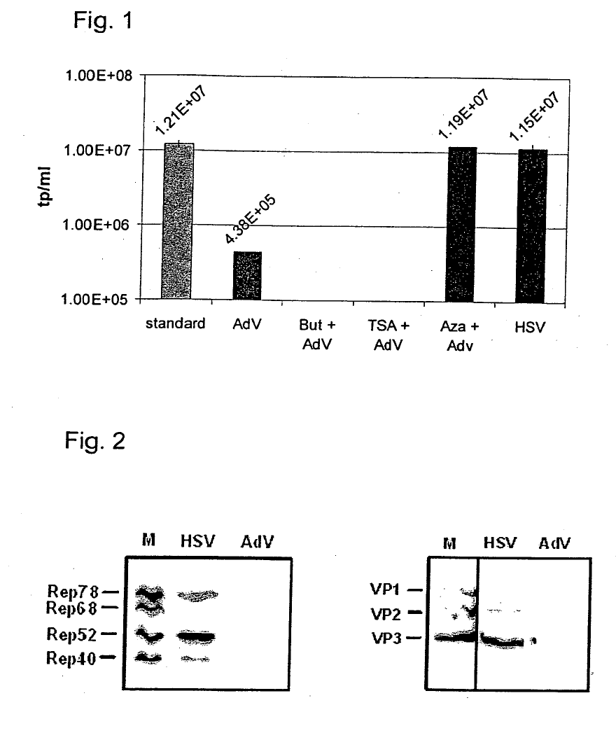

In order to establish whether there was an effect based on histone deacetylation or DNA methylation, the producer cells were treated with Na butyrate (But) (Sigma, Deisenhofen), which is, if anything, a nonspecific inhibitor of histone deacetylation, with the specific inhibitor of histone deacetylation trichostatin A (TSA) (Sigma, Deisenhofen) or with the DNA methylation inhibitor 5-azacytidine (Aza) (Sigma, Deisenhofen) (see FIG. 1). All the treated cells were also infected with adenovirus (AdV). In several experiments, it was shown that, while infection of the producer cells with adenoviruses, as helper viruses, resulted in a comparatively low yield of transducing rAAV particles (tP), transiently transfecting helper and vector constructs and infecting with adenovirus resulted in rAAV being produced with very good efficiency.

It was possible to restore this AAV packaging efficiency either by using HSV as helper virus or by using AdV in combination with Aza; by contrast, using But and TSA did not result in an increase in AAV packaging efficiency.

Apparently, DNA methylation suppresses the AAV genes, with it being possible to overcome this suppression by using HSV or using AdV in combination with a DNA methylation inhibitor but not by using AdV on its own.

### Example 3

### Level of Expression of Rep and Cap by Producer Cells Following Infection with HSV or AdV

A comparison of the expression of AAV-2 Rep and Cap by the integrated rep and cap genes in producer cells following HSV-1 or AdV infection showed clearly that, in contrast to HSV-1, AdV did not give rise to any significant expression of Rep and Cap (see FIG. 2). This once again demonstrates the suppression of the integrated AAV-2 genes.

In this experiment, 2E+06 cells were infected, per assay sample, with HSV-1 or adenovirus (in each case MOI 10). 48 h after infection, the lyzates were prepared, treated 1:1 with 2× protein buffer (100 mM Tris-Cl pH 8.0, 2 mM EDTA, 4% SDS, 20% glycerol, 10% beta-mercaptoethanol, 0.02% bromophenol blue), and boiled at 95° C. for 5 min. Identical quantities from each assay sample were separated by SDS polyacrylamide gel electrophoresis (SDS-PAGE). The semidry method was then used to transfer the samples to a nitrocellulose membrane. The samples were detected using the Rep-specific antibody 303.9 and the Cap-specific antibody B1 (Wistuba et al. (1997) J. Virol. 71:1341-1353).

1-21. (canceled)

22. A method for producing viral vectors which are derived from parvoviruses, said method comprises inhibiting the methylation of the viral vectors by a DNA methylation inhibitor.

23. The method as claimed in claim 22, wherein the DNA methylation inhibitor is selected from the group consisting of a nucleoside analog, a low molecular weight inhibitor, a DNA-derived direct inhibitor of DNA cytosine methyl transferase, an antisense oligonucleotide inhibitor of DNA cytosine methyl transferase, a herpesvirus, in particular an HSV, and herpesviral genes.

24. The method as claimed in claim 22, wherein the DNA methylation inhibitor is selected from the group consisting of 5-azacytidine, 5-azadeoxycytidine, 5-fluorocytosine, S-adenosylhomocysteine, and EGX30P.

25. The method as claimed in claim 22, wherein the viral vector is produced in a cell which contains the necessary genes for forming virus particles.

26. The method as claimed in claim 22, wherein the viral vector is produced by a vector cell.

27. The method as claimed in claim 22, wherein the viral vector is produced by a packaging cell or a producer cell.

28. The method as claimed in claim 27, wherein the packaging cell or the producer cell is stably transfected with helper constructs.

29. The method as claimed in claim 22, wherein not more than 50%, preferably not more than 20%, particularly preferably not more than 10%, of the methylation sites which are present in the viral vector which is produced are methylated.

30. The method as claimed in claim 22, wherein the DNA methylation inhibitor inhibits the methylation of the methylation sites which are present in the rep and cap genes or their promoters.

31. The method as claimed in claim 22, wherein a helper virus is additionally added for producing the viral vector.

32. The method as claimed in claim 31, wherein the DNA methylation inhibitor is used before the helper virus is added.

33. The method as claimed in claim 22, wherein the viral vector is derived from a dependovirus, particularly preferably from an adenoassociated virus (AAV).

34. The method as claimed in claim 33, wherein the viral vector is derived from an AAV selected from the group consisting of AAV-1, AAV-2, AAV-3, AAV-4, AAV-5, AAV-6, AAV-7, and AAV-8.

35. A method for producing a viral, parvovirus-derived vector in cell lines which are stably transfected with helper constructs, for the purpose of increasing the packaging efficiency at least 5-fold, especially 10-fold, preferably 20-fold and, in particular, 25-fold, as compared with producing the vector using an adenovirus without adding DNA methylation inhibitors, said method comprising inhibiting the methylation of the viral, parvovirus-derived vector by a herpesvirus.

36. The method as claimed in claim 35, wherein a cell as defined in claims 25 to 28 is used for producing the viral vector.

37. The method as claimed in claim 35, wherein the viral vector is derived from a parvovirus, particularly preferably from an AAV, in particular from an AAV selected from the group consisting of AAV-1, AAV-2, AAV-3, AAV-4, AAV-5, AAV-6, AAV-7, and AAV-8.

38. The method as claimed in claim 33, wherein for producing rAAV, a cell is used which contains at least one copy of a helper construct for expressing at least one AAV Rep protein and at least one AAV Cap protein and, in addition, at least one copy of a recombinant AAV plasmid which carries foreign DNA which is flanked by ITR regions.

39. The method as claimed in claim 38, wherein the nucleic acids which encode the Rep protein and the Cap protein are functionally separate and operatively linked to the natural AAV regulatory sequences.

40. A vector construct which is derived from parvoviruses, wherein not more than 50%, preferably not more than 20%, particularly preferably not more than 10%, of the methylation sites which are present are methylated.
